(19) United States
(12) Patent Application Publication
(10) Pub. No.: US 2007/0041865 A1
(43) Pub. Date: Feb. 22, 2007

(54) Electronic system and methods of dispensing a scent and antidote

(76) Inventors: Ramy P. Ayoub, Arlington Heights, IL (US); Robert F. D'Avello, Lake Zurich, IL (US); Emad S. Isaac, Woodridge, IL (US); Ryan E. Seick, Cary, IL (US)

Correspondence Address: MOTOROLA, INC., 1303 EAST ALGONQUIN ROAD, IL01/3RD, SCHAUMBURG, IL, 60196, US

(21) Appl. No.: 11/205,890

(22) Filed: Aug. 17, 2005

(51) Int. Cl. A61L 9/015

(52) U.S. Cl. 422/3; 422/5; 422/62; 422/123; 700/285

(57) ABSTRACT

A dispensing system in a vehicle comprises a dispensing device 22 enabled to provide a desired scent or antidote, a gas detection unit 24 enabled to detect undesirable gases and odors, and a controller 20 enabled to provide electrical controls to the dispensing device 22.

### FIELD OF INVENTION

The present invention relates to an electronic system and methods of dispensing a desired scent or an antidote in a controlled environment of a vehicle.

### BACKGROUND

Scent emanating devices are used in many controlled environments such as vehicles and bathrooms. Such devices are needed to combat foul or undesirable odors or even freshen the surrounding environment. These devices suffer from various shortcomings. They constantly render the same smell, degrade over time and often are difficult to obtain.

Present scent emanating devices are not able to combat noxious or harmful odors in a controlled manner. An example of the use of such devices is in vehicles. Passengers in a vehicle experience foul odors when they pass by a land-fill or a sewage treatment facility. There is a need to develop a better scent dispensing system and method, whereby people in controlled environments such as vehicle are not subject to foul gases for any amount of time.

### BRIEF DESCRIPTION OF THE DIAGRAMS

The accompanying figures together with the detailed description below are incorporated in and form part of the specification, serve to further illustrate various embodiments and to explain various principles and advantages all in accordance with the present invention.

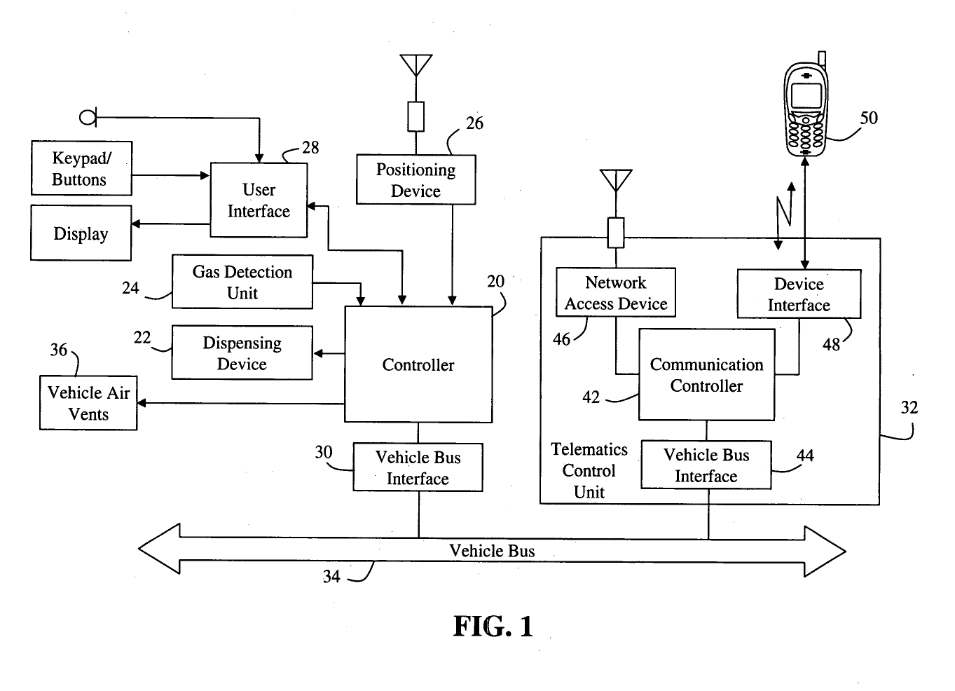

FIG. 1 depicts a block diagram of one embodiment of a scent dispensing system in a vehicle.

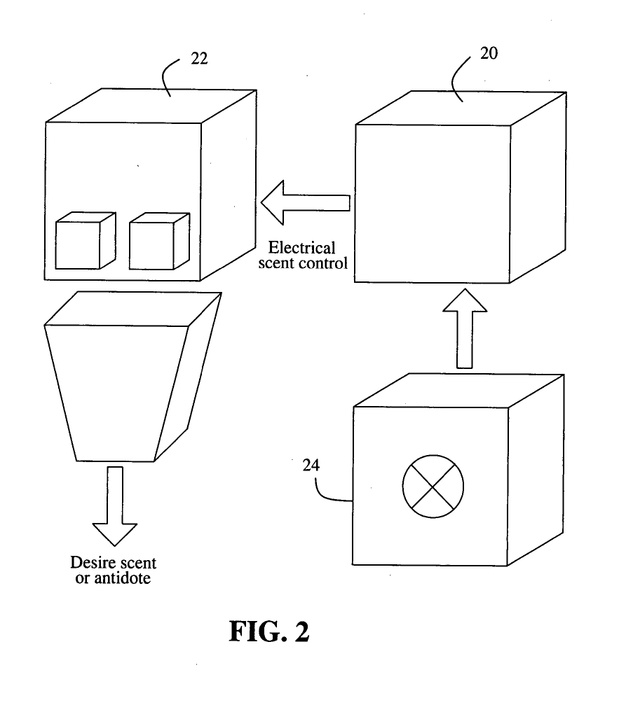

FIG. 2 discloses a schematic diagram of one embodiment of the main components of the scent dispensing system.

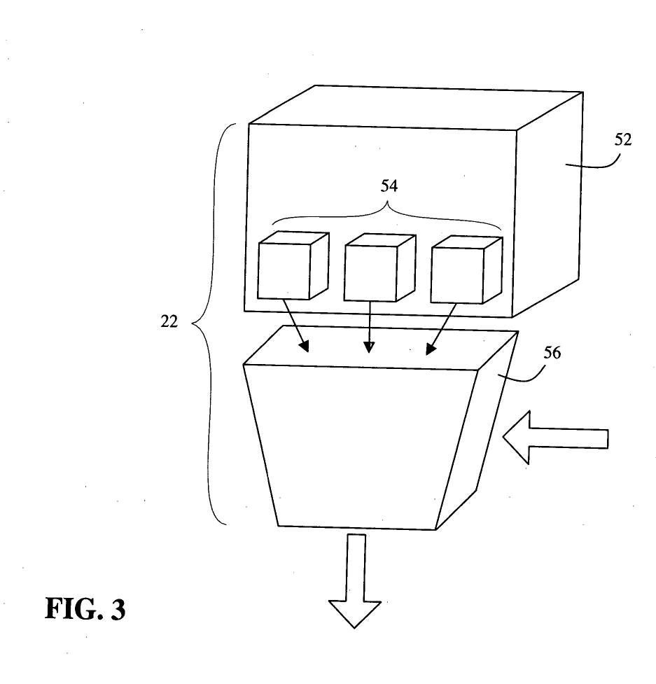

FIG. 3 discloses a schematic diagram of one embodiment of the dispensing device of the scent dispensing system.

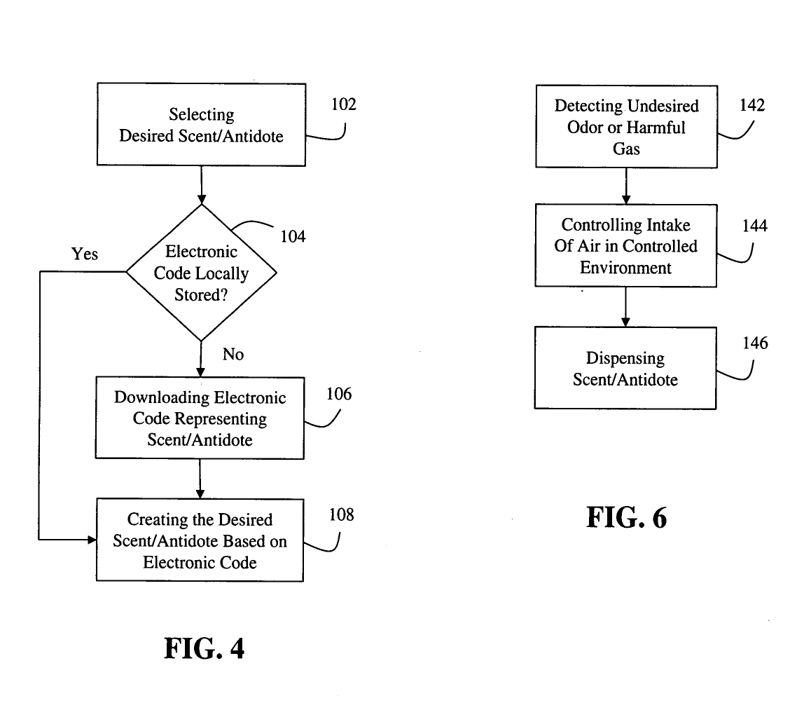

FIG. 4 depicts a flowchart of one embodiment for a method of acquiring and downloading a desired scent in a scent dispensing system.

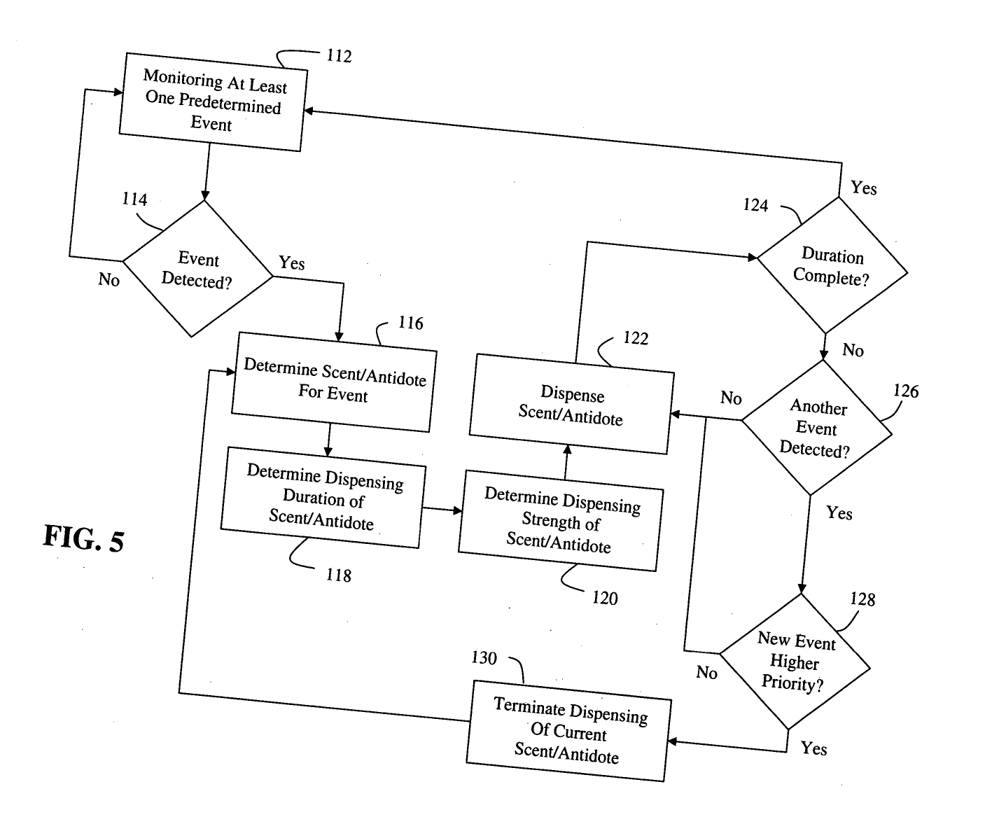

FIG. 5 depicts a flowchart of one embodiment for a method of dispensing a desired scent.

FIG. 6 depicts a flowchart of one embodiment for a method of combating an undesired odor or a harmful gas in a controlled environment.

### DETAILED DESCRIPTION OF THE INVENTION

The present invention may be embodied in several forms and manners. The description provided below and the drawings show exemplary embodiments of the invention. Those of skill in the art with the benefit of this disclosure will appreciate that the invention may be embodied in other forms and manners not shown below. The invention shall have the full scope of the claims and shall not be limited by the embodiments shown below. It is further understood that the use of relational term, if any, such as first, second, top and bottom, front and rear and the like are used solely for distinguishing one entity or action from another, without necessarily requiring or implying any such actual relationship or order between such entities or actions.

Scent emanating devices, which are available, render the same smell, degrade over time and are not easily available. While these devices can keep the environment fragrant, they are unable to combat sudden strong foul odors or gases. Therefore, people in such environments still experience foul odors and are susceptible to harmful gases. The present invention relates to a system and methods for dispensing a scent or an antidote to combat foul odors or harmful gases.

Turning now to FIG. 1, one embodiment of a scent dispensing system in a vehicle is shown. Although the system described herein is described in the context of vehicles, those skilled in the art with the benefit of this disclosure will realize that the present invention may also be applied to other controlled environments. Additionally, in the context of vehicles, the present invention may be applied to different types of vehicles including automobiles, buses, trains, and airplanes. In one embodiment, the scent dispensing system comprises of components such as a controller 20, a dispensing device 22, a gas detection unit 24, a positioning device 26, and a user interface 28. The scent dispensing system may further comprise of interfaces to other components in the vehicle such as a vehicle bus interface 30 that allows communication with a communication transceiver such as a Telematics control unit 32. As explained further below, an interface to a communication transceiver is advantageous because it allows access to additional scent and antidote electronic codes. In one embodiment, the vehicle bus interfaces 30 provides communication between the controller 20 and a Telematics control unit 32 over a vehicle bus 34. Depending on the configuration of the scent dispensing system, the controller 20 may also be able to manage vehicle components such as vehicle air vents 36.

Referring to FIGS. 1 and 2, the dispensing device 22 is configured to provide a desired scent within the cabin of the vehicle or other controlled environment. The gas detection unit 24 is configured to detect undesirable gases and odors within a specific environment. The controller 20 is configured to manage the system and provide electrical controls to the dispensing device 22 and other vehicle components. For instance, in one embodiment, upon detecting the presence of a foul odor or a harmful gas, the gas detection unit 24 provides electronic feedback to the controller 20. The controller 20 is then configured to trigger the dispensing device 22 with the information needed to dispense a particular scent corresponding to the information sent by the controller 20. For example, the controller 20 may receive feedback from the gas detection unit 24 regarding a need to dispense scent to counteract the foul odor of a local landfill. As disclosed, those skilled in the art shall appreciate that the feedback provided by the gas detection unit 24 can be based on the predetermined odors stored within the system. Alternatively, the controller 20 may be configured to monitor the location of the vehicle from information received from the positioning device 26. Based on the location of the vehicle and known landfills, the system and other communication systems may inform the controller 20 of the presence of a specific odor within the area.

In any event, the controller 20 responds to the feedback and triggers the dispensing device 22 with an electronic code corresponding to the scent or antidote for counteracting specific odors, such as those from a landfill. The dispensing device 22 may comprise basic scents and antidotes necessary to combine and produce a scent or antidote based on the electronic code received from the controller 20. For scents that are not present in the system, or not obtainable from a remote service provider, the user can be prompted for appropriate action. Similarly, the controller 20 can also take other counteracting steps—such as closing the vehicle air vents 36. Additionally, in other embodiments, the controller 20 may be configured to reverse the vehicle air vents 36 to force air out of the vehicle while dispensing the combative odor.

Under a further embodiment, the user may wish to have a different scent or antidote for his vehicle based on his user specified preferences. The system may be configured, through user interface 28, to accept instructions and user preferences through a keypad or a microphone. If a micro phone is used, then a voice recognition unit will need to be incorporated into the user interface 28.

In one embodiment, the system can be used as an air freshener for everyday use in a vehicle or any controlled environment. Different scents can be dispensed for each user of the controlled environment based on the user's preferences. Pursuant to this embodiment, the controller 20 can be configured to trigger the dispensing device 22 to dispense a desired scent corresponding the user preferences when the user uses the controlled environment. The controller 20 then provides electronic feedback to the dispensing device 24 to dispense the desired scent.

In yet a further embodiment, the system can be configured to receive additional scents or antidotes by downloading an electronic code through the vehicles wireless communication device such as the Telematics control unit 32 shown in FIG. 1. In that figure and in one embodiment, the Telematics control unit 32 is shown to include a communication controller 42, a vehicle bus interface 44, a network access device 46 and a device interface 48. The controller 20 may receive new or updated scent or antidote codes from a remote location from messages received by the network access device 46 or through a device interface 48 that is in communication with a portable wireless communication device 50. Any downloaded codes may be passed to the controller 20 by the communication controller 42 over the vehicle bus 34.

Referring to FIG. 3, in one embodiment, the dispensing device 22 may comprise a scent container 52, which stores a plurality of basic scents in a plurality of scent cartridges 54; and a scent mixer 56, which mixes the plurality of basic scents and dispenser, which provides the desired scent or antidote. The shape of the containers and dispenses are merely meant for ease of representation and seek to illustrate an embodiment of the present invention. The design of the system including the design of each individual containers, dispenses, ducts and other components can be modified based on predetermined preferences. Those skilled in the art shall appreciate that the number of basic scents can be increased based on the needs of the user to create additional scents or antidotes.

The scent mixer 56 can be triggered by manual user input or a predetermined event to mix and dispense the desired scent. For example, if a toxic gas was released in a controlled environment such as an automobile, a train or an airplane, the scent dispensing system would detect the presence of such a toxic gas send a signal to the scent mixer 56 to mix and dispense a desired antidote using an electronic code corresponding to the desired antidote. The scent dispensing system would then dispense the antidote to combat the particular toxic gas and neutralize the effect of the toxic gas. The scent mixer 56 may also control the concentration and resulting strength of the scent or antidote by controlling the volume by which the scent or antidote is dispensed. The scent concentration and dispensing duration are all controlled by the controller 20. The dispensing device 22 can have at least one antidote container (not shown) to provide the desired protection against foul and harmful odors such as tear gas, poisonous gases and the like.

According to an embodiment of the invention, as disclosed in FIG. 4, a method of acquiring and downloading a desired scent or antidote in a dispensing system comprises a step 102 of selecting a desired scent or antidote. For example, a driver can select one fragrance or antidote amongst various choices from a dispensing system, which can be installed in his vehicle, through the user interface 28 described above. In one embodiment, this selection can be a manual selection from a variety of choices through the user interface 28. The selection may also be automatic depending on predetermined conditions that may exist in the vehicle.

In any event, once a desired scent or antidote is selected, in step 104, the dispensing system then determines whether an electronic code associated with the desired scent or antidote is locally stored in the controller 20. If the electronic code associated with the desired scent or antidote is not locally stored in the controller 20, then in step 106 the controller 20 may request a download of an electronic code for the selected desired scent or antidote. As described above, in one embodiment, the scent dispensing system may be configured to connect to a remote location using a wireless communication transceiver such as the Telematics control unit 32. The wireless communication transceiver may communicate with a remote service provider to obtain the electronic code associated with the desired scent or antidote. The system may also be configured to allow users to purchase the scent or antidote from on-line stores selling scents or antidotes.

Once the appropriate electronic code is downloaded, or obtained locally in memory, the desired scent is created in step 108 as per the electronic code. According to another embodiment, the desired scent or antidote is created by combining or blending a plurality of basic scents or antidotes, which are stored in the dispensing system, based on the electronic code representing the desired strength. After combining the plurality of basic scents or antidotes to create the desired scent or antidote, the dispensing system dispenses the desired scent or antidote.

The scent dispensing system is also enabled to monitor a scent or antidote level in a basic scents or antidote cartridge, which stores a basic scent or antidote. The system determines whether or not the basic scents or antidote cartridge requires to be refilled. If the basic scents or antidote cartridge requires to be refilled, the system informs the user to refill the basic cartridge. The user interface 28 of the dispensing system can also allow the user to select the strength of the scent to be dispensed and the duration for dispensing the scent. This enables the user to have more control over the surrounding environment and a more pleasant and satisfactory experience.

According to another embodiment shown in FIG. 5, a method of dispensing a desired scent comprises a step 112 of monitoring at least one predetermined event in a controlled environment and then dispensing the desired scent or antidote based on the occurrence of the predetermined event in the controlled environment. Accordingly, in step 114, the controller 20 will make a determination of whether a predetermined event exists. The predetermined event can be a detectable event such as a vehicle ignition cycle, a geographic location and/or a time of day.

Once an event is detected, the controller 20 in step 116 will determine a desired scent or antidote for the event. As described above, in one embodiment, the controller 20 has access either through local memory or through a remote service provider a series of electronic codes associated with desired scents and antidotes.

In step 118, the controller 20 may further determine a dispensing duration of the desired scent or antidote. For example, the duration for dispensing a scent based on ignition cycle may be pre-preprogrammed to 10 seconds. In another example, the dispensing duration may be programmed to the duration of the detected event, such as noxious gases. The dispense duration may also be tied to specific geographic positions. The system is also enabled to dispense the desired scent for a predetermined time period. This predetermined time period depends on the strength a bad odor in the controlled environment. For example, if a vehicle is approaching a land-fill, a positioning system location can indicate to the scent dispensing system to initiate dispensing a desired scent to dispel the upcoming foul smell from the land-fill area. The scent can be dispensed for as long as the system detects the presence of foul smells or until the vehicle has moved out of the land-fill area, based on the location of the vehicle.

In step 120, the controller 20 may also determine a dispensing strength of the scent or antidote. For instance, the strength of the scent or antidote may be associated with the strength of the foul smell or harmful gas. The scent dispensing system is also enabled to detect small traces of foul smells and dispense the desired scent to combat such foul smells. At step 122, the scent or antidote is dispensed.

In step 124, the controller 20 may monitor the duration of the dispensing scent or antidote. When the duration is complete, the method returns to step 112 where the controller monitors the controlled environment for another predetermined event.

In a further embodiment of the present invention, the method includes the steps 126 and 128 where the controller 20 monitors whether another predetermined event occurs during the time another scent or antidote is being dispensed by the system. These further steps of the method allows for the prioritization of scent and antidote dispensing in accordance with multiple detected events. For example, if the vehicle is passing a local landfill and dispensing the associated combative scent, and then the system detects a harmful gas, the dispensing system will perform the step 130 of terminating the dispensing of the combative scent in favor of the antidote. If a higher priority event occurs, the method may then proceed back to step 116 where a new scent or antidote is determined based on the higher priority event.

According to another embodiment as shown in FIG. 6, a method of combating one of an undesired odor or harmful gas comprises the step 142 of detecting one of the undesired odor or harmful gas. Upon detecting the presence of either of the odor or the harmful gas, the dispensing system in step 144 then controls the intake of air in the controlled environment. In one embodiment, the system may isolate the vehicle cabin from outside air, it may regulate the air intake into the cabin, and/or the air flow out of the cabin, depending on the detected event. Thereafter, the system in step 146 dispenses a scent or antidote to combat either the undesired odor or the harmful gas.

Thus, the dispensing system described herein can be used to provide safety as well as avoid an unpleasant atmosphere. For example, if a toxic gas was released in a controlled environment such as an automobile, a train or an airplane, the dispensing system would detect the presence of such a toxic gas and control the flow of air in and out of the controlled environment. The dispensing system would then dispense the antidote to combat the particular toxic gas and neutralize the effect of the toxic gas. The dispensing system can be enabled to dispense the antidote in an amount based on the strength of the harmful gas or the undesirable odor. The dispensing system can also be enabled to dispense the antidote based on a plurality of regulatory guidelines needed to combat harmful gases.

What is claimed is:

1. A method of acquiring and downloading a desired scent or antidote into a dispensing system, the method comprising:
selecting a desired scent or antidote;
downloading an electronic code representing the desired scent or antidote;
creating the desired scent or antidote based on the electronic code.

2. The method of claim 1, wherein the creating step further comprises:
combining a plurality of basic scents or antidotes in the dispensing system based on the electronic code to give the desired scent or antidote; and,
dispensing the desired scent or antidote.

3. The method of claim 1, further comprising:
monitoring a scent level in a basic scent cartridge;
determining that the basic scent cartridge in the scent dispensing system requires refilling; and
informing a user about refilling the basic scent cartridge.

4. The method of claim 1 wherein the selecting step comprises manually selecting the desired scent via a user interface.

5. The method of claim 4, further comprising providing the user interface to the dispensing system to allow the user to select a duration of the desired scent or antidote and a desired strength.

6. A method of dispensing a desired scent, the method comprising steps of:
monitoring at least one predetermined event in a controlled environment; and
dispensing the desired scent based on the occurrence of the predetermined event in the controlled environment.

7. The method of claim 6, wherein the predetermined event comprises a detectable event, the detectable event comprising one of a vehicle ignition cycle, a geographic location, a time of day, a foul odor detected by a first gas sensor, and a harmful gas detected by a second gas sensor.

8. The method of claim 6, wherein the dispensing step further comprises of setting a dispense duration based on at least one of a type of the predetermined event or a magnitude of the predetermined event.

9. The method of claim 6, wherein the dispensing step further comprises of setting a dispense strength based on at least one of a type of the predetermined event or a magnitude of the predetermined event.

10. The method of claim 6, wherein the dispensing step further comprises:
dispensing the desired scent in an amount based on a predetermined criteria, wherein the predetermined criteria includes one of a strength of a bad odor, a user preference, a plurality of regulatory requirements, a plurality of guidelines, and a number of people in the controlled environment.

11. The method of claim 6, wherein the dispensing step further comprises:
monitoring a second predetermined event in a controlled environment during the occurrence of a first predetermined event; and
comparing a priority of the second predetermined event to a priority of the first predetermined event.

12. A method of combating one of an undesired odor and a harmful gas in a controlled environment, the method comprising:
detecting one of the undesired odor and the harmful gas; and
controlling the intake of air in the controlled environment.

13. The method of claim 12, wherein the dispensing step further comprises:
dispensing a scent or antidote based on one of the undesired odor and the harmful gas.

14. The method of claim 13 wherein an amount and duration of scent or antidote dispensed depends on a detected strength of one of the undesired odor and the harmful gas and on a duration of detection of one of the undesired odor and the harmful gas.

15. A dispensing system in a vehicle, the dispensing system comprising:
a dispensing device enabled to provide a desired scent or antidote;
a gas detection unit enabled to detect an undesirable gas or odor; and
a controller enabled to provide electrical controls to the dispensing device based on the detected undesirable gas or odor.

16. The dispensing system of claim 15 further comprising a positioning device to provide a location of the vehicle to the controller, the controller enabled to provide electrical controls to the dispensing device based on the location of the vehicle.

17. The system of claim 15, wherein the dispensing device comprises a scent container, which stores a plurality of basic scents in a plurality of scent cartridges; and a scent mixer, which mixes the plurality of basic scents and dispenser, which provides the desired scent.

18. The system of claim 15, wherein the dispensing device comprises at least one antidote container enabled to provide a desired protection.

19. A system for managing a cabin environment of a vehicle, the system comprising:
a positioning unit that determines a location of the vehicle;
a vent that provides a passage for air between the cabin environment and an exterior environment; and
a controller that controls the vent based on the location of the vehicle.

20. The system in claim 19 further comprising a dispensing device that is capable of delivering a desired scent or antidote within the cabin environment, the controller further capable of controlling the delivery of the desired scent based on the location of the vehicle.
